(19) United States
(12) Patent Application Publication
(10) Pub. No.: US 2025/0069707 A1
(43) Pub. Date: Feb. 27, 2025

(54) SYSTEMS AND METHODS FOR PROCESSING ELECTRONIC IMAGES TO ESTIMATE CARBON SEQUESTRATION

(71) Applicant: ACRE IO, LLC, The Plains, VA (US)

(72) Inventors: Dror HAOR, Hulda (IL); Hagit Liven BAR YEHUDA, Holon (IL); Gordon R. SMITH, Lake Forest Park, WA (US)

(73) Assignee: ACRE IO, LLC, The Plains, VA (US)

(21) Appl. No.: 18/799,598

(22) Filed: Aug. 9, 2024

Related U.S. Application Data
(60) Provisional application No. 63/519,047, filed on Aug. 11, 2023.

(51) Int. Cl. G16C 20/20; G16C 20/70

(52) U.S. Cl. CPC G16C 20/20; G16C 20/70

(57) ABSTRACT

Techniques for registering carbon sequestration include: receiving carbon sequestration data corresponding portions of a geographic region; comparing entries to a registry; assigning entries to clusters associated with carbon sequestration values; and determining a total carbon sequestration based on an aggregating of carbon sequestration values of the entries. In another aspect, a register-based carbon sequestration may utilize continuous learning, including generating estimates of carbon sequestration based on high-resolution aerial LiDAR, multispectral imagery, or the like. Unsupervised learning and a ground-based calibration procedure are usable to delineate distinct forest types within mixed forest area. Such a calibrated carbon sequestration system demonstrates superior accuracy compared to satellite-based analysis, and is able to estimate carbon sequestration on a grid, enabling generalization across multiple forest types and scales of aggregation within a unified framework.

### RELATED APPLICATIONS

The application is a non-provisional of, and claims the benefit of priority to, U.S. Provisional Application No. 63/519,047, filed on Aug. 11, 2023, the entirety of which is incorporated herein.

### TECHNICAL FIELD

Various embodiments of this disclosure relate generally to processing electronic images and, more particularly, to systems and methods for using machine-learning techniques to process electronic images to estimate carbon sequestrations.

### BACKGROUND

The role of forests as a significant mitigator of anthropogenic carbon dioxide (CO2) emissions is integral to the global efforts to combat climate change. Precise monitoring of carbon sequestration is a high priority for governments and organizations striving to achieve a zero atmospheric carbon balance. Remote sensing and machine learning has emerged are powerful tools for estimating Above-Ground Biomass (AGB) in diverse ecosystems. However, accurately measuring the carbon sequestrated by forests at various scales and forest types is still a technological and practical challenge carrying the risk of carbon over and/or underestimation, which may lead to wrong decision-making, flawed climate change mitigation actions, financial losses, and more.

This disclosure is directed to addressing above-referenced challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for processing electronic images, e.g., using machine learning techniques, to estimate carbon sequestration.

In one aspect, an exemplary embodiment of a computer-implemented method for registering carbon sequestration may include: receiving one or more entries of carbon sequestration data, each entry corresponding to a respective portion of a geographic region; comparing each entry to a feature space in a register of entries for the geographic region; based on the comparing, assigning each entry to a respective cluster amongst a plurality of clusters of entries in the register, each cluster of the plurality of clusters being associated with a respective carbon sequestration value; and determining a total carbon sequestration for the geographic region based on an aggregating of each carbon sequestration value of each entry in the geographic region.

In another aspect, an exemplary embodiment of a computer-implemented method for registering carbon sequestration may include: receiving one or more entries of carbon sequestration data, each entry corresponding to a respective portion of a geographic region, the geographic region including at least one sub-region, and each sub-region including at least one portion; comparing each entry to a feature space in a register of entries for the geographic region; based on the comparing, assigning each entry to a respective cluster amongst a plurality of clusters of entries in the register, each cluster of the plurality of clusters being associated with a respective carbon sequestration value; for each sub-region in the geographic region: aggregating all entries for each portion included in the sub-region; and determining a respective carbon sequestration value for the sub-region based on the respective carbon sequestration values assigned to each entry aggregated for the sub-region; and determining a total carbon sequestration for the geographic region based on the respective carbon sequestration value of each sub-region in the geographic region.

In a further aspect, an exemplary embodiments of a system for registering carbon sequestration may include: at least one memory storing instructions and a register of entries of carbon sequestration data for a geographical region, the geographic region including at least one sub-region, and each sub-region including at least one portion, each entry corresponding to a respective portion of the geographic region; and at least one processor operatively connected to the at least one memory, and configured to execute the instructions to perform operations. The operations may include: assigning each entry to a respective cluster amongst a plurality of clusters of entries in the register, each cluster of the plurality of clusters being associated with a respective carbon sequestration value; for each sub-region in the geographic region: aggregating all entries for each portion included in the sub-region; and determining a respective carbon sequestration value for the sub-region based on the respective carbon sequestration values assigned to each entry aggregated for the sub-region; and determining a total carbon sequestration for the geographic region based on the respective carbon sequestration value of each sub-region in the geographic region.

In another aspect, an exemplary embodiment of a method includes using a register-based carbon sequestration approach integrated within a continuous learning mechanism. In some embodiments, this method includes generating estimates of forest carbon sequestration based on one or more of high-resolution aerial LiDAR (for example, collected from an unmanned aerial drone or manned helicopter), multispectral imagery, or the like, which may be advantageous beyond the limitations of satellite-based imagery. The combination of unsupervised learning and a ground-based calibration procedure, such as in the systems and methods disclosed herein, are usable to obtain experimental results including delineation of distinct forest types, as many as ten or more, within a vast area of mixed forest spanning, in one embodiment, 55,000 hectares. The calibrated carbon sequestration estimation demonstrates superior accuracy compared to satellite-based analysis, as evidenced by rigorous cross-validation using a dataset of hundreds of ground-surveyed plots. Employing a continual learning mechanism, a system according to one or more embodiments disclosed herein is able to estimate carbon sequestration on a grid, for example measuring 25×25 meters, enabling generalization across multiple forest types and scales of aggregation within a unified framework.

Forests and other land vegetation play a vital role in mitigating anthropogenic CO2 emissions, sequestering 25%-30% of the annual global emissions through photosynthesis. The sequestrated CO2 is used by plants for growth, development, and reproduction but is also emitted back to the atmosphere during the respiration process. However, the woody biomass both below and above ground retains a large portion of this sequestrated CO2 as structural carbon, and quantifying the world's forested area AGB is of major importance for understanding forests' role in carbon sequestration, forest health, and development.

In one technique, the global forests biomass state at large scales may be quantified using satellite databases through remote sensing with databases such as the Global Ecosystem Dynamics Investigation (GEDI) and similar databases. For more regional and local scales, one practice for AGB estimation is the tree allometry approach, which empirically links the tree dimensions to its biomass. Ground measurements of tree geometry (stem Diameter at Breast Height (DBH) and occasionally also tree height) plugged into a species-specific allometric equation and up-scaled to the entire Area of Interest (AOI). By using the canopy empirical allometric relationship obtained rather accurately from airborne imaging, a decent relationship is shown between canopy diameter and height of individual trees to their AGB in various species.

The use of various methods of satellite output data analysis, whether it is LIDAR-based, multispectral images, or their combination, together with machine learning methods is a promising avenue in the AGB estimation, mostly in the individual species or forest type. However, using satellite and ground sampling to capture the year-to-year changes in AGB and achieving accurate numbers for the entire AOI is a challenging task and may lead to large inaccuracies. Such inaccuracies are common in mature forests, with complex and dense canopy, forest gaps, biomass change due to tree mortality or regeneration, etc. Both approaches may overlook such phenomena, due to resolution or scale limitations. The need to accurately measure and account for the AGB state and dynamics in large forested areas is crucial for understanding and quantifying the potential of a certain forested area to reduce atmospheric CO2 concentrations. This need becomes more urgent in recent years due to the emerging carbon offset market, allowing entities to offset their carbon emissions by purchasing carbon credits from forested areas (i.e., their owners) and compensating for their carbon emissions. The carbon credit market growth has raised concerns about overestimation and misrepresentation of biomass and biomass change, which could lead to environmental and economic consequences. While not all of this growth would count as carbon credits according to one analysis, 11.86 gigatons of CO2 warming equivalent (GtCO2e) was calculated and credited in 2022 via various sources and regulators, representing 23.17% of global greenhouse gas emissions. As a result, there has been an increased effort by various national and international entities to accurately account for forests' carbon credits and improve the accuracy and credibility of those measurements. Current methods for quantifying forest biomass using remote sensing generally have large errors when applied to small AOIs. Furthermore, as the carbon offset market evolves, there will be a higher demand for high accuracy in smaller AOIs. This will enable higher visibility of forest areas that are affected by weather conditions such as floods and fires, and by man-made changes, such as deforestation and harvesting.

To pursue this effort, scientists and forest managers are seeking ways to improve the efficiency and accuracy of AGB measurement and monitoring. In one technique, as discussed above AGB may be estimated using ground-based measurement of tree geometry, and species-specific allometric equations that are up-scaled to large forested areas. With the improvement of remote sensing (airborne and satellite-based) technologies in the last two decades there is an increasing amount of data that allow better spatial coverage of a specific AOI, developing methods to estimate AGB from remote sensing-based datasets, occasionally validated by ground measurements. The most common remote sensing method used for AGB estimation is based on the measurement of both spectral reflectance data and Light Detection and Ranging (LiDAR) data. Both spectral reflectance and LiDAR data can be collected from various sources, such as satellites, airborne, and ground-based sensors. Both methods are effective in estimating AGB to some extent but limited in their accuracy and their ability to cover complex and varying forest types. Furthermore, these methods are limited by the spatial and temporal resolution and availability of the data.

With remote sensing tools, the forest biomass quantity and dynamics can be estimated as a function of multiple features. Some features are direct measures, such as height, DBH, canopy cover, and crown diameter, and some are indirect proxies of AGB dynamics as the Normalized Difference Vegetation Index (NDVI) and the Green Leaf Index (GLI) as a feature for carbon uptake and AGB gain, e.g., based on comparisons taken periodically or over time. Remote sensing data may be used to obtain ample information that can describe those features. Some features can be calculated through explicit formulas, such as multispectral markers or mean LiDAR point-cloud height, etc. Other features, such as tree coverage, or the number of trees, are impossible to describe by an explicit formula. This disclosure proposes systems and methods for detecting such features via machine learning methods. However, conventional machine-learning methods are unable to achieve high accuracy in complex features, and for that, a more flexible and comprehensive method is needed. In some embodiments, a machine learning approach includes using deep supervised learning for AGB estimation. Deep learning (DL), as a class of machine learning algorithms, may be used to automatically extract features from raw data. Alternatively, unsupervised machine learning may be used to find repetitive patterns in unlabeled datasets. These patterns, or a combination of feature sets, in embodiments, may be used to reduce the amount of labeled data, and thus enable tackling existing problems that weren't possible with supervised learning alone. Combining remote sensing and machine learning techniques has the potential of providing a powerful tool for estimating the amount of carbon stored in complex forests through the detection of AGB highly correlative features.

In various embodiments, this disclosure pertains to systems and methods for carbon sequestration estimation using machine learning from remote sensing data, combined with a continuous learning mechanism. In one aspect, a method combines thorough and large-scale ground measurements with spectral reflectance data from airborne imagery with machine learning techniques to classify different types of forest cover and estimate their AGB with high accuracy. In an embodiment, a Register-based Carbon Capture and Storage (CCS) procedure is used to the uncertainties and heterogeneity of complex forests by establishing a register comprised of a set of categories (clusters) and utilizing continual learning to enable generalization to any forest types and biomes via a ground-based calibration process.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1A depicts an image of a geographical distribution of a study area.

FIG. 1B depicts a detail view of the study area from FIG. 1A.

FIG. 2 depicts an exemplary embodiment of a nested plot ground sampling design for the study area from FIG. 1A.

FIG. 3A depicts a bar graph of tree height for the study from FIG. 1A.

FIG. 3B depicts a bar graph of tree diameter for the study from FIG. 1A.

FIG. 4 depicts a block diagram of an exemplary embodiment of a data preprocessing stage.

FIG. 5 depicts a block diagram of an exemplary embodiment of continuous register learning system for carbon sequestration estimation.

FIG. 6 illustrates an exemplary embodiment of a sample matching operation mechanism that may be performed by a machine-learning-based matcher.

FIG. 7 illustrates features extracted from images taken in the study from FIG. 1A.

FIG. 8 illustrates an exemplary embodiment of a tree segmentation model for the continuous register learning system of FIG. 5.

FIG. 9 depicts an example Receiver Operating Curve (ROC) from validation of the tree segmentation model of FIG. 8.

FIG. 10 depicts an example chart of Principle Component Analysis components for the tree segmentation model of FIG. 8.

FIG. 11 depicts an example chart of features and weights from the Principle Component Analysis of FIG. 10.

FIG. 12 depicts an example chart of correlation and importance of features from the Principle Component Analysis of FIG. 10.

FIG. 13 depicts an example bar graph for evaluating clustering techniques, according to one or more embodiments.

FIG. 14 depicts a scatter plot of carbon sequestration distributions for measured ground plots from the study of FIG. 1A.

FIG. 15 illustrates exemplary categories of data determined by for the study from FIG. 1A.

FIG. 16 depicts a graph of an error between calculated and determined sequestration rate for each of the categories from FIG. 15, according to techniques discussed herein.

FIG. 17 depicts an example scatter-plot of expected error per area of interest for the study from FIG. 1A.

FIG. 18A illustrates a region categorized into distinct groups, according to one or more embodiments.

FIG. 18B illustrates an exemplary detail view of a portion of the region from FIG. 18A.

FIG. 18C illustrates reference images corresponding to the portion of the region from FIG. 18B.

FIG. 19 depicts exemplary images from the study from FIG. 1A.

FIG. 20 illustrates an exemplary environment 100 for carbon sequestration estimation.

FIG. 21 is a simplified functional block diagram of a computer, according to one or more embodiments discussed herein.

### DETAILED DESCRIPTION OF EMBODIMENTS

Reference to any particular activity is provided in this disclosure only for convenience and not intended to limit the disclosure. A person of ordinary skill in the art would recognize that the concepts underlying the disclosed devices and methods may be utilized in any suitable activity. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of +10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

Below, various systems, methods, operations, and techniques are described with reference to an experimental study, along with corresponding experimental results. However, it should be understood that, in various embodiments, systems, methods, operations, and techniques incorporating one or more aspects of this disclosure may be incorporated into any suitable activity.

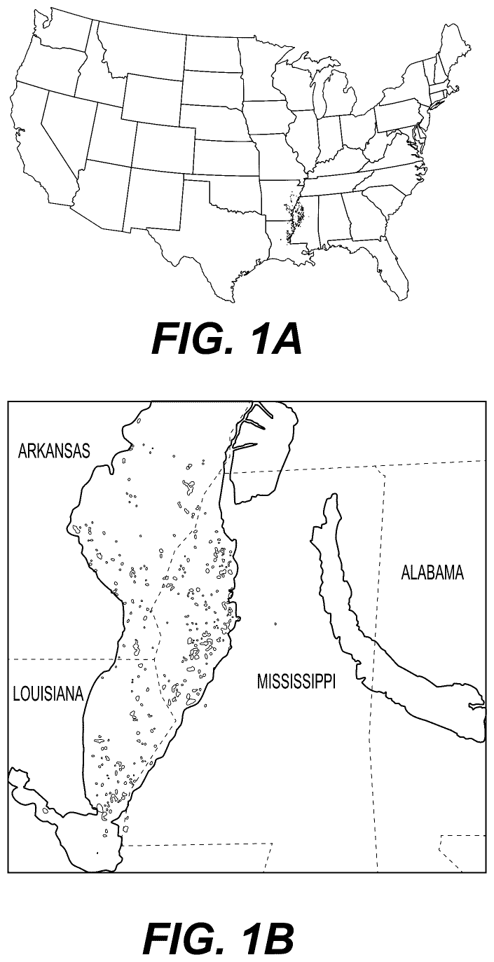

The exemplary study area discussed herein for ground truthing and validation of the Register-based Carbon Sequestration (CCS) method is located in the southeastern United States, encompassing parts of Mississippi (MS), Louisiana (LA), and Arkansas (AR) (FIG. 1), consisting of 55,000 hectares of afforested land, with a mixed deciduous tree composition.

FIGS. 1A and 1B depict a geographic distribution of the exemplary study area. In red are areas along the Mississippi Basin that comprise the area. In FIG. 1A, the general location of the study area overlaid on a map of the continental United States. As illustrated in the zoomed-in view of FIG. 1B, the study area includes varied forest types across multiple states. Each dot represents one area of interest (AOI) studied and measured in these examples.

The study area includes 657 forested parceled lands, denoted here as Tracts. The size of the sub-tracts ranged from 6 to 1416 hectares per tract, with a median size of 84.2 hectares. The majority of the tracts (97%) are located along the Mississippi River basin, between latitudes 30° and 36° and longitudes 90° and 93° with mean elevation of 45 (±20) meters above sea level.

The dominant species planted in these tracts is Southern Red Oak (Quercus falcata var. falcata) following a smaller amount of White Oak (Quercus alba) and Eastern Cottonwood (Populus deltoides), Green Ash (Fraxinus pennsylvanica), and Sweet Gum (Liquidambar styraciflua). For the entire tree inventory at the study area and species distribution, see table 1.

Table 1 includes a list of tree species that were planted in the exemplary study sites and their respective wood density. Species-specific AGB from ground sampling may be calculated in accordance with their tree size class. The AGB may then be up-scaled using the specific species distribution as found in the ground survey and planting densities. ‘Miscellaneous’ refers to all non-planted and randomly developed species that are not in the planting list but were found in the survey. The species portion is based on the ground data collection, as discussed in further detail below.

TABLE 1

| | | | Species |
|---|---|---|---|
| Common | Scientific | Wood | proportion |
| Name | Name | Density* | (ground survey) |
| Ash, green | Fraxinus pennsylvanica | 0.53 | 6.8% |
| Box Elder | Acer negundo | 0.44 | NA |
| Black Gum | Nyssa sylvatica | 0.46 | NA |
| Cottonwood, eastern | Populus deltoides | 0.37 | 11.7% |
| Cypress, bald | Taxodium distichum | 0.42 | 1.3% |
| Elm, American | Ulmus americana | 0.46 | NA |
| Hackberry | Celtis occidentalis | 0.49 | NA |
| Hickory | Carya spp. | 0.64 | 0.3% |
| Locust, Honey | Gleditsia triacanthos | 0.6 | NA |
| Maple, Silver | Acer saccharinum L. | 0.47 | NA |
| Oak, Swamp White | Quercus bicolor | 0.64 | 3.6% |
| Oak, Southern Red | Quercus falcata | 0.52 | 52.6% |
| Pecan | Carya illinoinensis | 0.6 | NA |
| Persimmon | Diospyros virginiana | 0.64 | 0.3% |
| Pine, Loblolly | Pinus taeda | 0.47 | 2.3% |
| Pine, longleaf | Pinus palustris | 0.54 | NA |
| Sweet Gun | Liquidambar styraciflua | 0.46 | 5.2% |
| Sycamore, American | Platanus occidentalis | 0.46 | 0.9% |
| Willow | Salix spp. | 0.36 | 5.2% |
| Miscellaneous | — | 0.52 | 9.9% |
| Mean wood | — | 0.50 (0.018) | — |
| density (±SE) | | | |

For ground data collection, a total of 802 ground sampling plots in this example, may be used for ground truth validation in two distinct rounds. The first exemplary round, here comprising 474 plots, may serve the purpose of initial register calibration and evaluation. In a second round, in an example here consisting of an additional 328 plots, unstable areas that lacked a sufficient number of ground plots during the initial calibration step may be re-calibrated. Additionally, the second round may be utilized to enhance the accuracy of other categories and refine the categories' representative values.

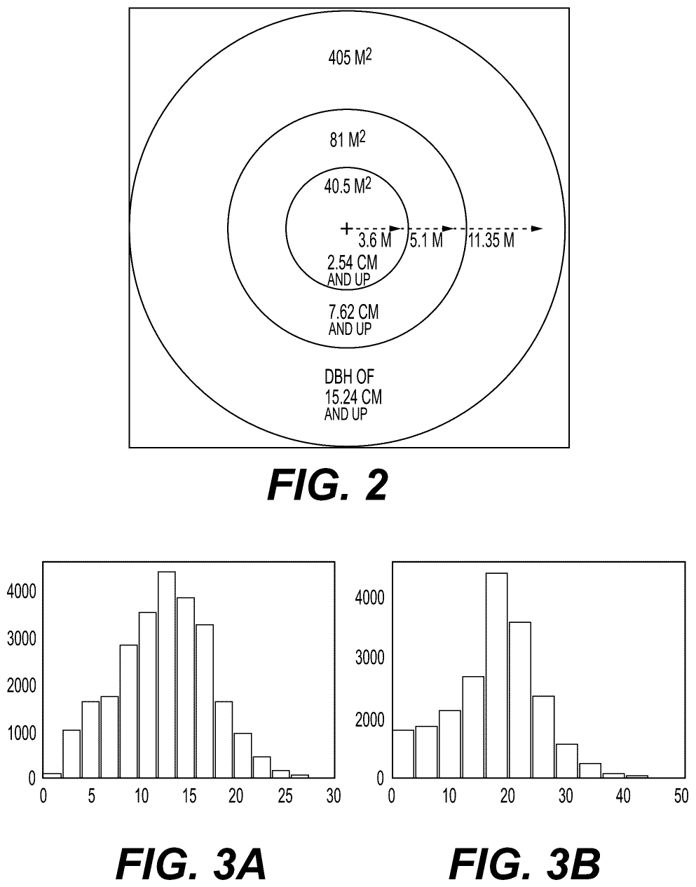

Measurements may be taken alongside the aerial survey period, and included tree species, height, and Diameter at Breast Height (DBH). A random method may be used for selecting sampling plot locations which may have constraints such as: (a) a minimal predetermined distance, for example of 20 meters, from the outer borders of the forested area, and (b) accessibility for ground teams. To facilitate sampling fieldwork, a circular nested plot design may be used. As illustrated in FIG. 2, the sampling area may be divided into three overlapping centered plots, which may be used for measuring different tree categories according to a DBH threshold.

FIG. 2 depicts a nested plot ground sampling design, indicating the area (in meters squared), the radii (in meters) and minimum tree diameter at breast height (in centimeters) to be measured on each subplot. It should be understood that the use of a nested plot is exemplary only, and that any suitable type of plot may be used.

In this nested plot, all trees with DBH>2.54 cm (1″) may be measured in the inner 40.5 m2 circle. In the 81 m2 circle only trees with DBH>7.62 cm (3″) may be included, and in the 405 m2 circle only trees with DBH>15.24 cm (6″) may be sampled. However, it should be understood that any suitable area may be evaluated in various embodiments. Inventory data may be delivered as a list of trees measurement collected within each plot, along with the plot center GPS position. In total in this example, 11,164 trees were measured, varying across both height and DBH ranges (FIG. 3), and together with the large species variation (Table 1) the ground sampling area and tree size and species, is a good representation.

FIG. 3A depicts an exemplary bar graph in which the horizontal axis measures tree height in meters, and the vertical axis measures a count of trees for each height for the 28,584 tree measurements collected as part of these examples. FIG. 3B depicts an exemplary bar graph in which the horizontal axis measures DBH in centimeters for the same tree measurements.

For aerial data collection, an aerial data collection campaign may be conducted, for example during the leaf-on season through October and November 2022, covering a net study area of 55,000 hectares and a 30-meter buffer around tracts borders. Data collection may include LiDAR and 4-channel multi-spectral RGB and NIR (RGBN) imagery. LiDAR data may be collected using the RIEGL VQ-1560II system (Riegl Inc., Austria) at a predetermined average altitude, for example 2,000 feet, with a predetermined spatial 2D resolution, for example of 50 points per square meter. The Red, Green, Blue, and NIR (near infra-red) (RGBN) imagery may be collected using the Phase One iXM-RS150F system (PhaseOne, Denmark) with a predetermined spatial resolution, for example of 5 cm/pixel.

Images may have a predetermined overlap, for example of 15%, between flight strips and 60% within strips. The location information for the imagery, including XYZ positioning and Yaw-Pitch-Roll payload readings, may be delivered as a CSV file, although many data formats are possible. Image-mosaic ground accuracy may be approximately five meters RMS and RGBN-to-LiDAR alignment accuracy of about one meter. As LiDAR is collected per flight strip, the collected point clouds of overlapping strips may be calibrated to match ground level by manually detecting the ground level per strip and aligning them on the stitched (overlapping) area. The calibrated raw LiDAR data and raw imagery may be denoted as raw data.

To perform carbon calculations, data from the ground survey (including DBH, height, and species) may be plugged into relevant allometric equations adjusted to tree size and species-specific equations for calculating the AGB per area. Using the AGB output, a CO2 equivalent per hectare (in units of tCO2e/hectare) may also be calculated, converting biomass to carbon (50% of the wood biomass) and using the stoichiometric conversion factor of 3.67 (44/12) to obtain CO2e. For each plot, plot carbon sequestration (Cplot in units of tCO2e/hectare) may be calculated, which is a measure of the total sequestration for that plot and may be calculated by equation (1):

$${C}_{plot} = {\sum }_{i}^{K} {C}_{i} / {A}_{i} \qquad ( 1 )$$

where K is the number of trees sampled in the plots, Cᵢ is the ith tree carbon sequestration and Aᵢ denotes the corresponding tree's circular centered-plots area, with possible values of 0.01, 0.02, 0.1 acres. This list of 802 plot carbon sequestrations may serve as ground truth data for the next analysis steps. As such, these plots are also referred to as “labeled data”.

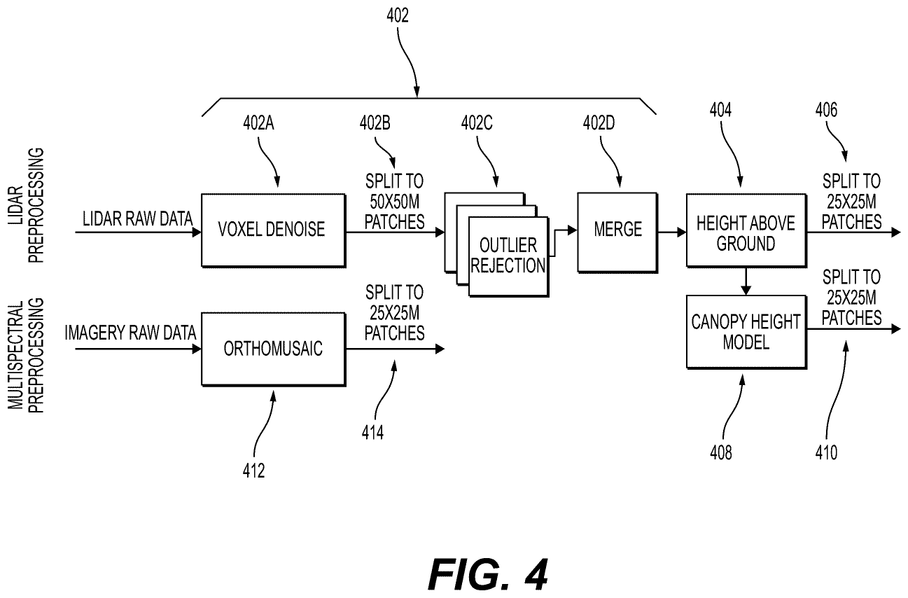

In a pre-processing step, a set of transformations may be performed on the raw data to simplify feature extraction, and to serve all subsequent stages optimally. The data preprocessing steps are described in two phases, as multispectral imagery (RGBN) and LiDAR-based point clouds are treated differently (FIG. 4).

FIG. 4 depicts a block diagram of the data preprocessing stages 400. At stage 402, LiDAR raw data may be cleaned from outliers and artifacts, and then at stage 404 may be normalized to represent height above ground. More specifically, raw LIDAR data may be collected as sets of point clouds in “strips” of flights. To preprocess the data, coarse noise removal may be performed on the point cloud, which involves eliminating dominant clusters of noise. This may be accomplished through a series of operations, which are referred to herein as the Voxel denoise stage 402a. In an exemplary embodiment, steps involved in this stage 402a may include any of the following:
1. The input point cloud is split into 1×1×1 m voxels;
2. Each voxel is represented by the number of points it contains;
3. Based on the new representation, 3D Connected Components (CCs) are computed, excluding empty voxels;
4. CCs elements which are composed of less than a 100 voxels are filtered out;
5. Angles between the center of mass of all components and a main component (biggest one) relative to the XY plane are determined;
6. Components with angles greater than 10′ are considered artifacts and are removed; and/or
7. Points are removed from artifact components.

After the coarse noise removal process at stage 402a, the point cloud may be divided into 50×50 m patches at stage 402b. Each patch may undergo finer outlier rejection at stage 402c, e.g., using the Point Data Abstraction Library (PDAL) Python package. In this embodiment, the statistical outlier filtering technique may be used, setting the filtering parameters to take a neighborhood of one cubic meter and a variance multiplier of two cubic meters. At stage 402d, the split patches may then be recombined to match their original position in the input.

The next exemplary stage 404 is height normalization, such that the height of each point may be represented by its exact distance from the ground. This may be achieved through the following steps using PDAL functions:
1. A Cloth Simulation Filter function is used to classify ground points; and/or
2. A Height above ground Nearest Neighbor function takes classified ground points and outputs normalized heights of trees relative to that ground.

At stage 406, the resulting point cloud may then be cropped/divided into patches of a predetermined size, for example 25×25 m, to facilitate further feature extraction, when the patch geometry, including location and vertices, may be the same as the RGBN data.

At stage 408, a canopy height model (CHM) may be generated based on the preprocessed LiDAR data, e.g., to simplify subsequent post-processing and analysis steps. The CHM, in this embodiment, may be built by interpolating only the points belonging to the first LiDAR return. An Inverse Distance Weighting (IDW) interpolation method may be used, e.g., with a resolution of 0.1 m/pixel to create the CHM. At stage 410, the resulting CHM may also be cropped into patches of the predetermined size, for example 25×25 m, in the same manner as the LiDAR and RGBN data, for further analysis in subsequent stages.

At stage 412, multispectral images may be fused together to produce a map. In an example, raw RGBN images may undergo preprocessing as a set of 4-channel images, along with their corresponding projection parameters to generate an orthomosaic. To achieve this, in some embodiments, a projective transformation may be performed on each image, converting it from the image plane to the world plane. These projected images may then be merged to produce a single map, which forms the orthomosaic. At stage 414, the orthomosaic may be divided into patches of a predetermined size, for example 25×25 m.

In various embodiments, a register-based carbon sequestration (CCS) method may be used. As used herein, a CCS method is or includes a learning method that enables users or automated systems to continuously define and adjust a register of forest types, while continuously keeping it up-to-date for a plurality of regions. One motivation for doing so is the understanding that remote sensing signals from different forest types, or even from the same forest type in different seasons, with the same carbon quantities, may vary significantly. To address the large variation between different geographies, species, and seasons, machine learning may be employed in a continual or periodic sequence. Machine learning methods according to this disclosure have the ability to find optimal weights for different features and fit them to desired target data. Combining machine learning with a continuous learning scheme as disclosed herein forms the ability to automatically adapt and optimize the system performance to any new input data.

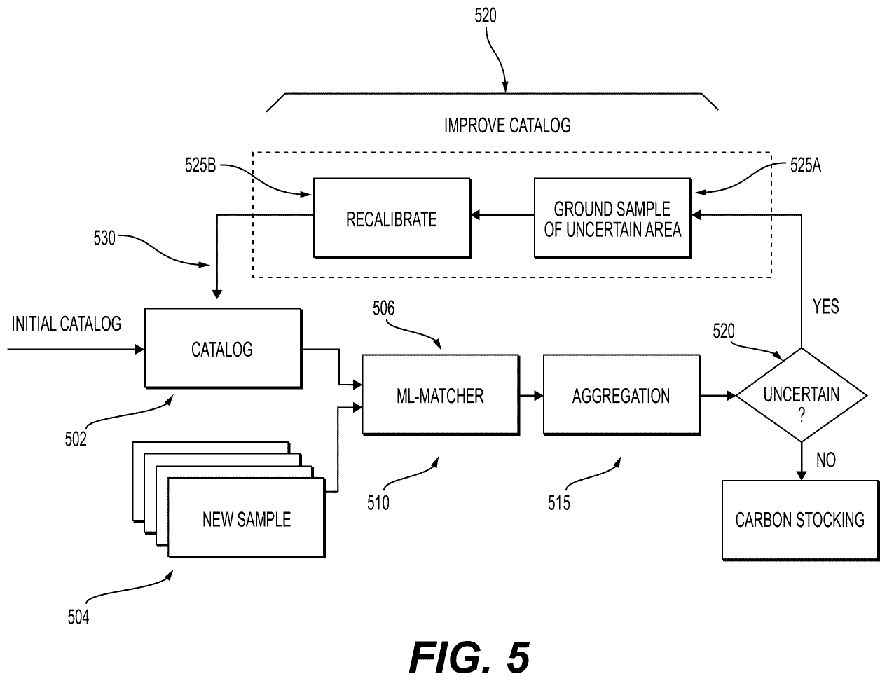

FIG. 5 depicts a block diagram of a continuous register learning system for carbon sequestration estimation. In particular, the scheme presented in FIG. 5 illustrates a situation in which some initial register 502 has already been generated, and new data 504 has been pre-processed and cropped to patches, treated as new samples.

A new sample/entry 504 may be defined as stacked patches of multispectral, LiDAR point cloud, and CHM data of a predetermined size, for example 25×25 m. At stage 510, each new sample 504 may be matched to its best fitting cluster in the register 502 via the ML-Matcher 506. Then, at stage 515, aggregation may be done on some pre-defined area of interest. At stage 520, the area of interest may be evaluated for certainty, e.g., via evaluating what portion of the area is known with at least a threshold certainty. For example, if an uncertain portion in that area is lower than 5 percent (e.g., “No” in stage 520), carbon sequestration for that area may be set. Alternatively (e.g., “Yes” in stage 520), the area is selected for re-calibration at stage 525, in which the uncertain portion is ground sampled at stage 525a, and the register is re-calibrated at 525b. As illustrated at stage 530, the routine repeats, e.g., until convergence. Further aspects of this scheme 500 are discussed below.

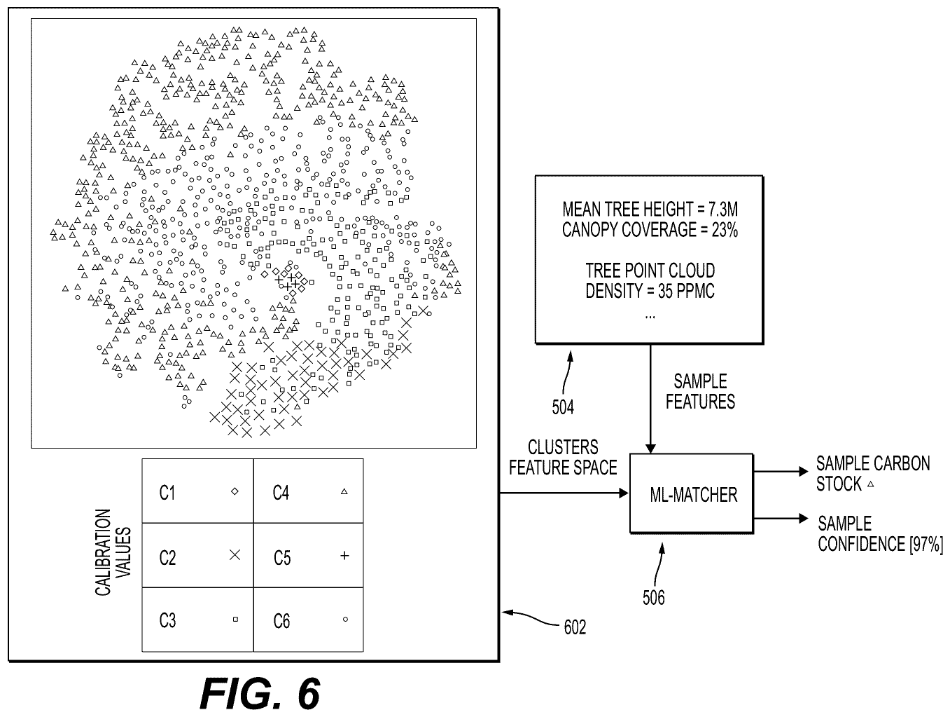

FIG. 6 illustrates a sample matching operation mechanism that may be performed by the ML-Matcher 506 of FIG. 5. The ML-matcher may compare each new sample 504 to a pre-generated feature space in the register 502, finding the best-fitting cluster alongside its confidence value. If the confidence value is above a predetermined threshold, e.g., 50% the sample carbon estimation is set as the best-fitting category carbon sequestration. Otherwise, it may be defined as an uncertain sample. The scatter plot 602 may be a 2D t-distribution Stochastic Neighbor Embedding (TSNE) visualization of a 6-clusters feature space.

The ML-Matcher 506 may obtain or access an already calibrated register 502, where its features, categories, and the carbon sequestration per category may already be defined. The ML-Matcher 506 may compare an unseen sample 504 with each of the categories in the register 502 to select the new sample 504's best-fit category and assign it to the category carbon sequestration, alongside a confidence level number, which in this embodiment is based on a predefined probabilistic mechanism. Then, as discussed above at stage 515 in FIG. 5, all samples may be aggregated over a designated AOI.

As noted above with regard to stage 520, if a large area of the AOI is defined as low-confidence (or “uncertain”), this area may be designated for the ground sampling process of stage 525 to collect data as discussed above. Once the ground campaign is done, the register 502 may be retrained, and the evaluation of the register 502 may be reiterated. If the uncertain area does not pass a certain pre-defined threshold, the ground campaign might not be needed. In this case the aggregation step returns AOI-level carbon sequestration as the average of all the samples within that AOI. The process may be iterative and automatic, and as the ML-Matcher 506 re-calibrates itself every iteration, it ensures convergence, which enables scalable solutions in the carbon sequestration domain.

Feature extraction may simplify the input data by identifying a set of representative features that are relevant to the problem at hand. This may be achieved by transforming the data into a lower dimensional space. For developing the CCS method, a set of features were assembled that would capture the connection between remote sensing data and the aggregated biomass of the area. The entire set of features (90 features in total), extracted from different data sources (LiDAR point cloud, CHM, RGBN imagery, and general metadata) is summarized in Table 2. The table presents different types of features which are extracted by statistical methods, straight-forward math calculations, or deep learning-based methods.

TABLE 2

| Source | Feature | | |
|---|---|---|---|
| data | type | Feature | Description |
| LiDAR | Vertical | max, min, mean, | height metrics of |
| | | cubic mean | all points |
| LiDAR | Vertical | std, mean/median | Point cloud height |
| | | absolute deviation | deviations |
| LiDAR | Vertical | Height percentiles | Feature |
| | | [25, 50, 75, 90] | |
| LiDAR | Vertical | skew, kurtosis, Inter- | Height |
| | | quartile range. | distribution |
| | | | metric |
| LiDAR | Density | density-m², | 2D/3D point cloud |
| | | density-m³ | density |
| LiDAR | Intensity | intensity mean/std | LiDAR return |
| | | | intensity |
| | | | distribution |
| CHM | Texture | Gray-Level | [53] |
| | | Co-Occurrence | |
| | | Matrix^{a} | |
| RGBN | Spectral | vari-mean, vari-std | Visible |
| | index | | Atmospherically |
| | | | Resistant |
| | | | Index(VARI) |
| | | | mean/std |
| RGBN | Spectral | gli-mean, gli-std | Green Leaf |
| | index | | Index (GLI) |
| | | | mean/std |
| RGBN | Spectral | vig-mean, vig-std | Green Vegetation |
| | index | | Index (VIgreen) |
| | | | mean/std |
| RGBN | Spectral | bi-mean, bi-std | Brightness Index |
| | index | | (BI) mean/std |
| RGBN | Spectral | ndvi-mean, ndvi-std | Normalised Difference |
| | index | | Vegetation Index |
| | | | (NDVI) mean/std |
| RGBN | Spectral | gndvi-mean, gndvi-std | Green Normalised |
| | index | | Difference |
| | | | Vegetation Index |
| | | | (GNDVI) mean/std |
| RGBN | Spectral | sr-mean, sr-std | Simple Ratio (SR) |
| | index | | mean/std |
| CHM | Tree | percent of tree coverage | Simple Ratio (SR) |
| | coverage | | |
| General | Age | forest planting age | Current year − |
| | | | Planting year |

^{a}The matrix include the following features: angular_second_moment, contrast, correlation, sum_of_squares_variance, inverse_difference_moment, sum_average, sum_variance, sum_entropy, entropy, difference_variance, difference_entropy, information_measure_of_correlation_1, information_measure_of_correlation_2

Table 2 includes a summary of the extracted features for each patch. For each data source, one or more feature types may be extracted, and for each feature type, several features may be extracted. In some embodiments, the features are manually engineered or selected to extract the most relevant information from the source data.

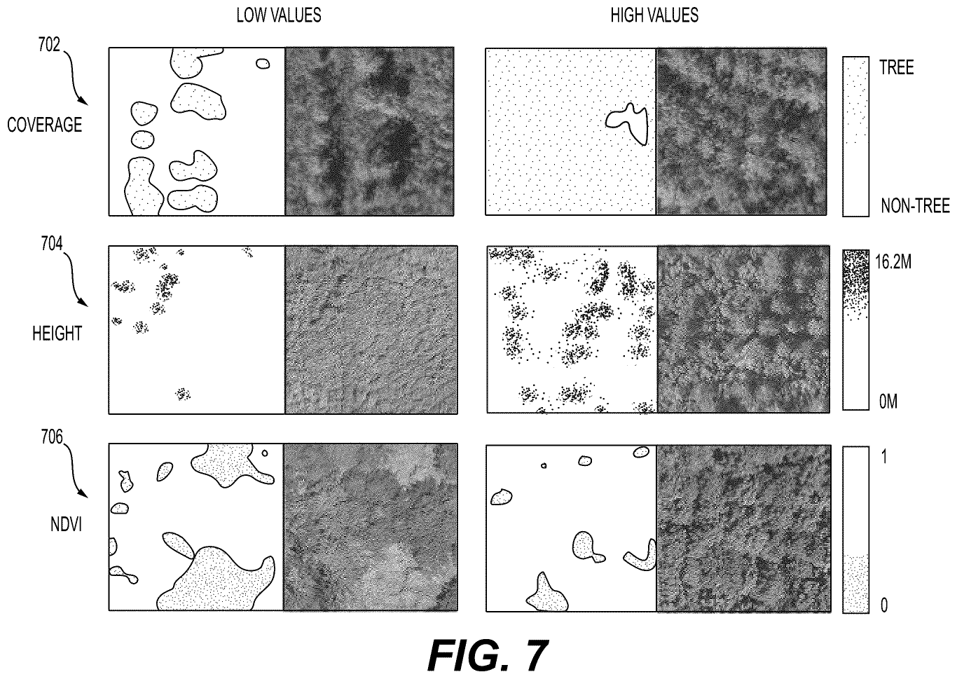

FIG. 7 depicts an example visual illustration of features extracted from photographs taken in a ground campaign. High and low values examples are given for coverage 702, height 704, and NDVI features 706. In each example, a pair of images shown on the left-hand side illustrates the feature distribution within a patch, and on the right-hand side, the raw image of that patch for reference. In the feature distribution images, the coverage feature is illustrated as a white area, the height as heat-map where the high tree tops are highlighted.

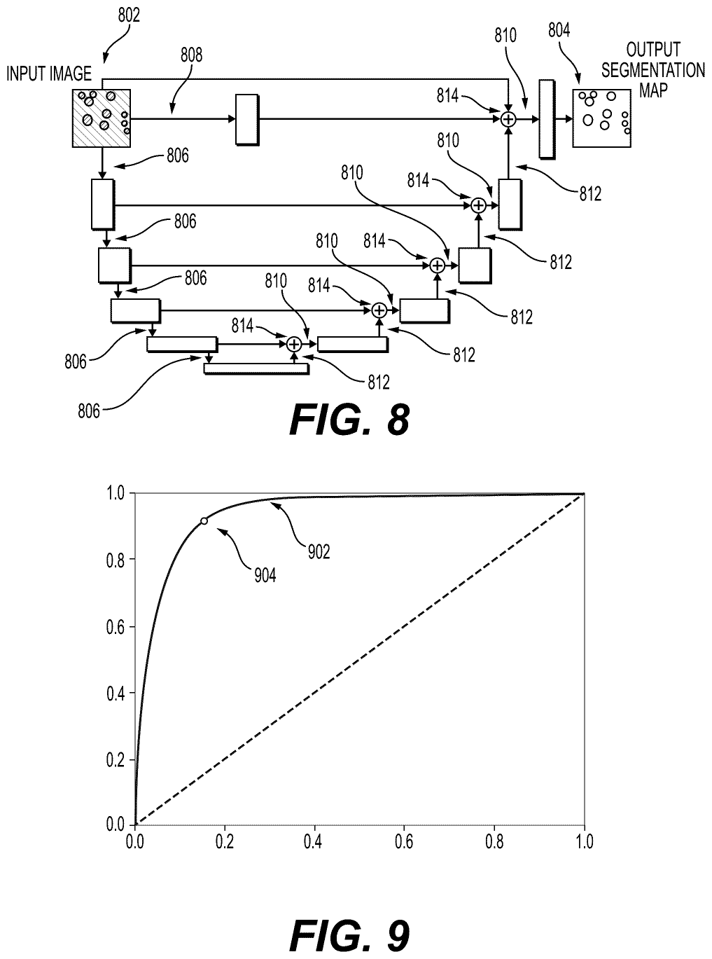

In some embodiments, aspects of tree coverage features may be determined via deep learning. Tree coverage may be defined as the percent/portion of ground area covered by tree canopies. Tree coverage information is important to separate different types of forests, and maintain an accurate carbon estimation on the patch level. Achieving accurate tree coverage calculations requires a clear distinction of trees from other elements, such as ground, short vegetation, shrubs, and crops. To address this necessity, in an exemplary embodiment, a deep learning segmentation model may be trained, for example based on a U-net architecture with a ResNet base and concurrent spatial and channel ‘squeeze & excitation’ (csSE) blocks as demonstrated in FIG. 8.

FIG. 8 depicts a tree segmentation model, for example based on the U-net architecture, and incorporates residual blocks and concurrent spatial and channel ‘squeeze & excitation’ (csSE). The input 802 to the model may be a Canopy Height Model (CHM) of a forest patch, and the output 804 may be a probability map of each input pixel to represent a tree-covered area. As illustrated in FIG. 8, the model may include ResNet blocks with scSE 806, a convolutional layer 808, upsampling layers 810, decoder layers 812, and/or concatenation blocks 814.

During the training stage, the model may be fed images of a predetermined size(s), for example 256×256 pixels, from the CHM in which each pixel is 0.1 m×0.1 m. The example dataset included 12,782 images, and each training image may be manually labeled to assign each pixel a value (1 for tree, 0 for non-tree). Here, these labels may be used as ground truth for training and validating the model. The dataset may be split into training, validation, and test sets, with predetermined ratios of, for example, 0.7, 0.1, and 0.2, respectively. The model may perform pixel-wise classification, determine the probability for each pixel pᵢ to be a tree, with the output pᵢ E [0, 1]. The objective function for model training may comprise a weighted sum of Binary Cross Entropy (BCE) and Dice Similarity Coefficient (DCS):

$$L = α {L}_{BCE} + ( 1 - α ) {L}_{DCS} \qquad ( 2 )$$

$$where$$

$${L}_{BCE} = - \frac{1}{N} {\sum }_{i = 1}^{n} {y}_{i} ln {p}_{i} + ( 1 - {y}_{i} ) ln ( 1 - {p}_{i} ) \qquad ( 3 )$$

$$and$$

$${L}_{DSC} = 1 - \frac{2 {\sum }_{i = 1}^{N} {p}_{i} {y}_{i}}{{\sum }_{i = 1}^{N} {p}_{i} + {\sum }_{i = 1}^{N} {y}_{i}} \qquad ( 4 )$$

where yᵢ is the ground truth of each pixel in a given sample. To further improve the training set, it was augmented with various transformations: rotation, flip, scaling, and masking. Next, the model may be tested on an independent test set, unseen by the model during the training process, and the predictions may be compared to the ground truth. A receiver operating curve (ROC) may be generated, as shown in FIG. 9, and used to determine the optimal threshold for classifying a pixel as either tree or no tree.

FIG. 9 depicts a Receiver Operating Curve (ROC) based on the validation set predictions of the tree segmentation model, in which the horizontal axis pertains to a rate of false positive identifications of a pixel as a tree, and the vertical axis reflects the true positive rate in the test data. The model may achieve an area under the ROC curve 902 (AUC) of 0.94. The classification threshold, denoted by the dot 904, may result in the best trade-off between TPR and FPR, for example of 0.42.

An example threshold of 0.42 was determined to give the best ratio between the false positive rate (FPR) and true positive rate (TPR). The tree coverage may be calculated as the percentage/portion of the area classified as tree of the total area within a given sample. The model's performance may then be tested using multiple types of input, for example RGB aerial images and/or CHMs. During training, the RGB-based model may present better performance than the CHM model, with an area under the ROC of 0.96 compared to 0.94 achieved by the CHM model. However, a manual inspection of non-annotated samples may reveal that the CHM-based model showed better generalization ability than the RGB-based model.

Feature selection may help improve model accuracy, prevent over-fitting, and reduce training time in machine learning models. In some embodiments, training the model includes a combination of supervised and unsupervised feature selection methods to identify the most relevant features. Supervised methods, such as a random forest (RF) regression model, may use the known target variable to guide the selection of features and tend to be more accurate and reliable, but require a sufficient amount of labeled data. Unsupervised methods, such as the Principal Component Analysis (PCA), might not use the target variable to guide feature selection and can be applied to large amounts of unlabeled data, but might not provide information about the feature-target association as in supervised methods.

Due to the limited amount of available labeled data by ground measurements, in some embodiments, a combination of supervised and unsupervised feature selection methods may be employed to identify the most relevant features for the clustering model. The RF model may be used, with measured CO2e values from labeled ground plots as the target variable, to guide the selection of features. In addition, unsupervised PCA may be used on the calculated features of unlabeled data to identify additional valuable features. By combining both approaches, it is possible to effectively utilize both labeled and unlabeled data to identify a comprehensive set of relevant features for the model. This allowed optimization of the accuracy and reliability of the model, despite the limited amount of the labeled data.

Every given (natural or planted) forest can be divided into more homogeneous forest types via unsupervised clustering. The clustering method enables the assignment of a specific CO2e value to all samples within a cluster. In addition to category allocation, it may be beneficial to report the level of confidence, or certainty, that a specific patch belongs to its assigned category. The determination of the confidence level is dependent upon the chosen clustering technique, with distance-based methods utilizing distance measurements to cluster centers, and probabilistic methods utilizing probability distributions to define the likelihood of a sample belonging to each cluster.

The objective of the Clustering Model Selection procedure is to identify and evaluate clustering methods for creating groups of patches based on similar features and assigning representative carbon values based on measured ground plots to each group. To achieve this, any suitable clustering process may be used. As illustrative examples, three different clustering algorithms may be compared and evaluated: K-Means (centroid-based), Birch (connectivity-based), and GMM (distribution-based), for several categories amounts. Standard clustering evaluation metrics, such as Akaike information criterion (AIC) and Bayesian information criterion (BIC) may be misleading when applied to clustering methods of different types. For example, some metrics may be better suited for evaluating clusters based on distances, while others may be more appropriate for evaluating clusters based on probability. As such, in order to have a fair comparison between different types of clustering methods, a custom evaluation metric may be used to accurately assess the uniformity of the resulting clusters and the uniformity and representation of AGB values within them.

One of the metrics that may be used for evaluation is mean Homogeneity Variance (HV). HV is a metric that assesses the uniformity of samples within clusters by quantifying the mean standard error for each feature in each category. The metric may be based on the coefficient of variation, which measures the variability of a series of numbers independently of the unit of measurement used for these numbers. Thus, the coefficient of variation can be used to compare distributions obtained with different units after scaling, when a lower HV value indicates a higher degree of uniformity. The HV metric can be applied at different levels of granularity, depending on the evaluation purpose. It can be used to measure HV at the feature level (HV_{ci,fj}), category level (HV_{ci}), or model level (HV_{model}). More specifically, given category cᵢ ∈{c₁, c₂, . . . , cₙ} and feature fⱼ ∈{f₁, f₂, . . . , fₘ} it is possible to define:

$${HV}_{{c}_{i} , {f}_{j}} = \frac{{σ}_{{c}_{i} , {f}_{j}}}{{μ}_{{c}_{i} , {f}_{j}}} , {HV}_{{c}_{i}} = \frac{1}{m} {\sum }_{j = 1}^{m} {HV}_{{c}_{i} , {f}_{j}} , {HV}_{model} = \frac{1}{n} {\sum }_{i = 1}^{n} {HV}_{{c}_{i}} \qquad ( 5 )$$

In a similar manner to mean Homogeneity Variance (HV), an additional independent metric may be used, called AGB Homogeneity Variance (AGB-HV), for evaluating the uniformity of AGB within categories. AGB-HV may focus on the target feature of AGB, which is not used in the clustering process, and evaluates its homogeneity within the cluster. AGB homogeneity is the expected property of a good cluster, as it reflects specific forest type with a small deviation of carbon values. AGB-HV may also be defined on category-level (AGBHV_{ci}) and model-level (AGBHV_{model}). When building model-level ground plot-based evaluation metrics, it is important to take into account the number of available measured ground plots per category. That is, stable clusters with a sufficient number of measured plots should be given a higher weight as defined here:

$${AGBHV}_{{c}_{i}} = \frac{{σ}_{{c}_{i} , AGB}}{{μ}_{{c}_{i} , AGB}} , {AGBHV}_{model} = {\sum }_{i = 1}^{n} \frac{{GP}_{{c}_{i}}}{GP} {AGBHV}_{{c}_{i}} \qquad ( 6 )$$

where GP_{ci} indicates the number of measured ground plots per category ci, and GP indicates the total number of measured ground plots.

After defining a model for stable and uniform clusters, each cluster may be calibrated individually. This calibration process entails assigning a specific biomass value to each cluster, which is known as the Cluster Calibration Value (CCV). Once a CCV is assigned to a particular cluster, all patches assigned to that cluster will be treated as having the same CCV. This calibration procedure ensures consistency and enables accurate biomass estimation within each cluster.

The calibration procedure may comprise one or more of the following steps:
1. Assign all the ground sample patches to their appropriate cluster using an already trained clustering model;
2. For each cluster, split all the training data into K equally sized groups and repeat K times per group;
(a) Select group j as the test group and combine all other K-1 groups as the train group;
(b) Find the optimal CCV with minimal mean carbon sequestration error for the train group by grid search (grid split by 0.01 tCO2e increments); and/or
(c) Test the mean carbon sequestration error on test group j:

$${err}_{c}^{j} = \frac{1}{L} {\sum }_{i}^{L} ( {p}_{g} - {p}_{p} ) .$$

where p_{g} is the patch ground truth value and pp is the patch predicted value according to the category CCV defined in (b), and L is the number of patches in the group.
3. Set cluster CCV by the mean of the K optimal CCVs from step (2)—this is the calibration step.
4. Assign each cluster a mean carbon sequestration error of

$${err}_{c}^{} = \frac{1}{M} {\sum }_{j}^{M} {err}_{c}^{j} .$$

5. Mark any cluster that has a total train and test group number of plots lower than a threshold number of plots, e.g., ten, as an unstable cluster.
6. Calculate the tract-level aggregated expected error for all tracts as

$${err}_{tract}^{} = {\sum }_{c}^{C} {err}_{c}^{tract} \cdot {w}_{c}^{{co}_{2}}$$

where err^{tract}_{c} is the sum of a category errors err_{c} across all patches within the tract and w^{co2}_{c} is the error weight within the tract for that category, calculated by Eq. (7).

$${w}_{c}^{{co}_{2}} = {\sum }_{{p}_{c}} {p}_{c}^{{co}_{2}} / {\sum }_{{p}_{tract}} {p}_{c}^{{co}_{2}} \qquad ( 7 )$$

where p_{c} and p_{tract} are the lists of all the patches within the category and the tract, respectively, and p^{co2}_{c} are the patch estimated carbon sequestration according to the category c calibration value.

The calibration procedure, as described above, may assign an optimal CCV to each cluster, ensuring high accuracy in carbon sequestration prediction. The mean carbon sequestration error for each cluster may also be calculated, allowing the identification of unstable clusters. The tract-level aggregated expected error may then be calculated as a measure of the overall accuracy of the method.

One aspect of the proposed CCS method is its use of uncertainty measures to detect unfamiliar types of forests plugged into the system. When a new sample is assigned to a cluster, it is given a carbon sequestration estimation as well as an uncertainty measure, which indicates the confidence level of the sample's similarity to other members of its assigned cluster. In an embodiment, a single type of confidence measure may be selected for each of the aforementioned clustering algorithms. For GMM, a probability density function based confidence measure may be implemented, for similarity estimation a bootstrapping approach may be used along with inferring confidence, and for the used K-means approach a confidence interval may be applied for the distance between a new sample point and its assigned centroid. In addition to calculating the uncertainty measure for each patch, a search may also be performed for geographically clustered areas of uncertainty to identify unfamiliar forest types. To do this, the project area may be divided into parcels of a predetermined size, for example 400×400 m, and the number of uncertain patches may be counted in each parcel. If a parcel has more than a predetermined number/portion/percentage of uncertain patches, for example 50%, it may be defined as an uncertain parcel, and all of its uncertain patches may be saved as an uncertain cluster to be sampled during the ground campaign. Results of the example embodiment above are discussed below.

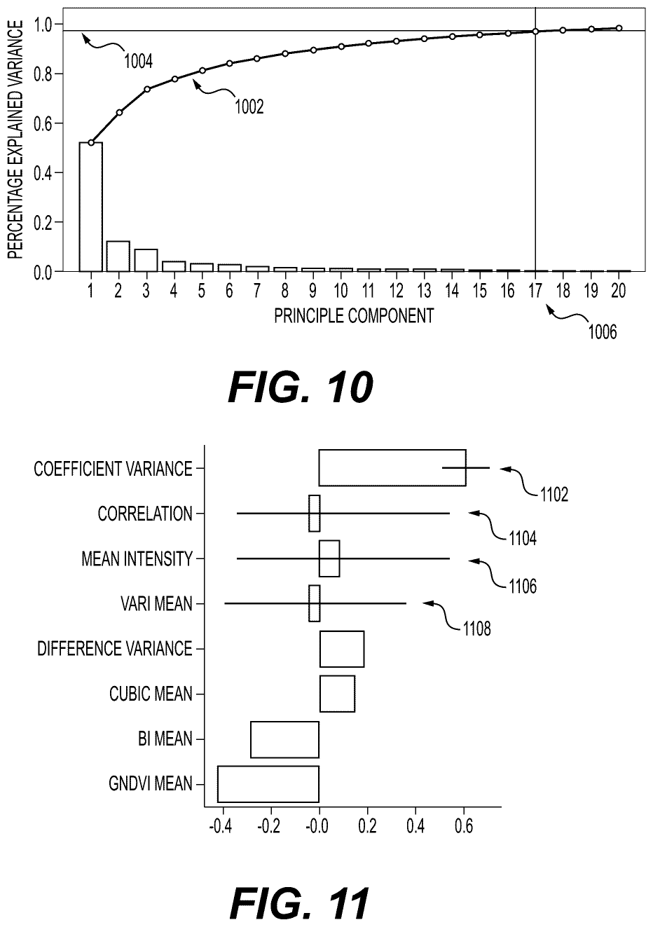

FIG. 10 depicts example PCA components for explained cumulative variance, in which the horizontal axis represents different principle components as determined via the PCA analysis, and the vertical axis represents the percentage by which each principle component explains the observed variance. The line 1002 tracks the running percentage of variance accounted for by the principle components, for example the horizontal line 1004 defines a value of 95.8% on the vertical axis for the 17th principle component 1006. As illustrated in FIG. 10, a predetermined number, for example 17 components, may found to be sufficient in explaining a high percentage, for example 95.8%, of the cumulative variance Principal Components.

A combination of tree height, texture, and spectral characteristics may be used as the initial set of eight top-scoring features out of these components. In an example, spectral features such as BI, GNDVI, and VARI may contribute to utilizing category separations and capturing essential information for clustering.

FIG. 11 depicts example Top Features and Mean Weights According to PCA. The x-axis displays the mean PCA weights of the leading feature from each principal component, with negative weights indicating a negative correlation. Large (either positive or negative) weights indicate that a variable has a strong effect on that principal component. Coefficient Variance 1102, Correlation 1104, Mean Intensity 1106, and VARI mean 1108 are features of two principal components, with their weights range visualized by respective lines and their mean weights depicted by the respective solid bars. Bars without black line are for features that were prominent in only one principal component.

Furthermore, the RF analysis of features importance revealed that height-related and height correlated features, including cubic mean, sum average, and height percentiles, may have a significant influence on tCO2e estimation, with feature-importance values>0.1.

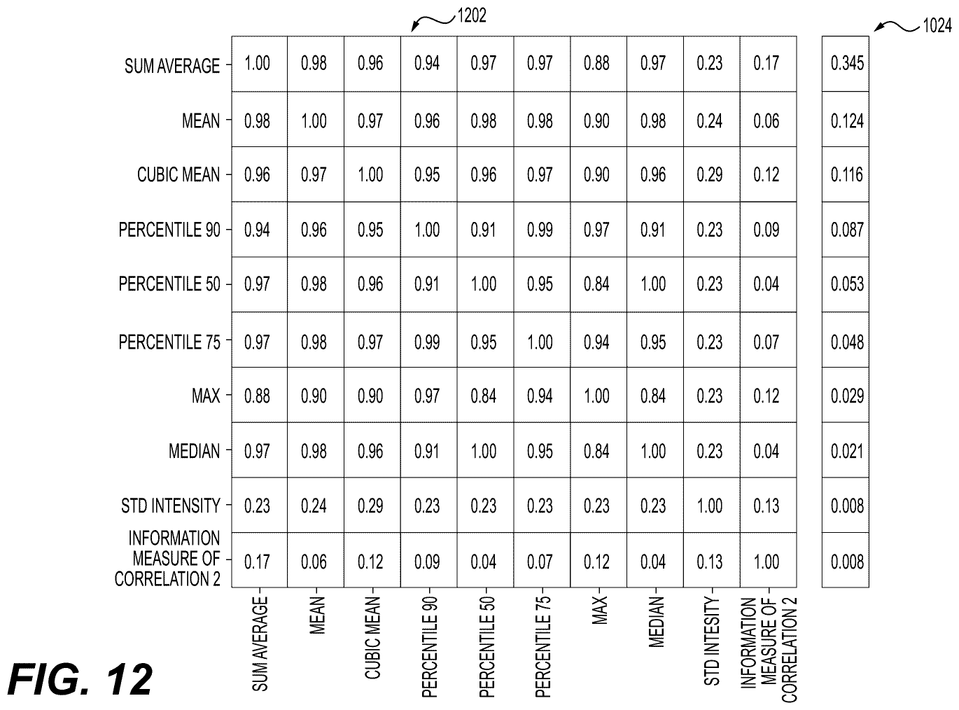

FIG. 12 depicts an example Correlation Matrix 1202 and a Feature Importance Scores Bar 1204. The rows and columns respectively represent the top ten features prioritized based on their impact on tCO2e, according to Random Forest Regression features' importance analysis. The matrix 1202 may be visualized as a heatmap of pair correlation values, and the score bar 1204 represents the feature importance scores as calculated from the model. In this example, a high correlation was observed among 8 out of the 10 features, and notably, the most prioritized feature-sum average, is among the correlated features. Hence, in an exemplary embodiment, this feature was selected as a representative for the final features set.

Among the most influential features, which may have the highest explanatory feature, with a feature importance score of 0.34, is the sum average from the texture feature type. Sum average measures the relationship between occurrences of pairs with lower intensity values and occurrences of pairs with higher intensity values. Since the sum average demonstrates a high correlation with the other leading features identified through the RF analysis, it may be chosen to complement the final feature set utilized for clustering, for example as listed in table 3

TABLE 3

| Feature-type | Feature | Equation |
|---|---|---|
| Height | Cubic mean | ${\sum }_{i}^{N} \sqrt[3]{{z}_{i}}$ |
| Height | Coefficient Variance | Σᵢ₌₁ⁿ (z_{i -} μ)²/Σᵢ^{N} zᵢ |
| Texture | Sum Average | [53] |
| Texture | Difference Variance | [53] |
| Texture | Correlation | [53] |
| Spectral | VARI | (G − R)/(G + R − B) |
| Spectral | NDVI | (NIR − R)/(NIR + R) |
| Spectral | Bright Index | \sqrt{R/G} |
| Intensity | Intensity | $\frac{1}{N} {\sum }_{i = 1}^{N} {I}_{i}$ |

Table 3 depicts an exemplary embodiment of selected features used for the register generation, classified by height, texture, and spectral characteristics, as listed in Table 2.

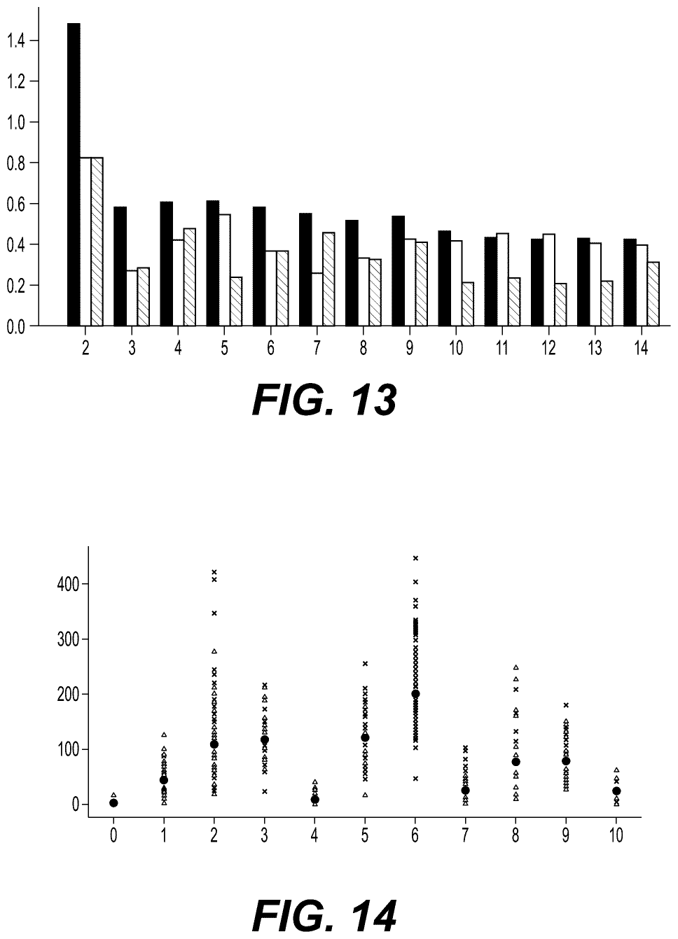

A comparison of three clustering methods (Birch, K-means, and GMM) across cluster numbers from 2 to 14 indicates that GMM outperformed Birch and K-means in terms of a composite metric combining mean Homogeneity Variance (HV) and AGB Homogeneity Variance (AGB-HV), as shown on FIG. 13.

FIG. 13 depicts a Clustering methods evaluation bar graph. Three clustering methods were evaluated-Birch (black), K-means (while), and GMM (hatched). Results of a composite metric of HV and AGB-HV (vertical axis) are given as a function of the number of clusters (horizontal axis). In this example, bar height represents composite scores for each method for each number of clusters, where a low score means more homogeneous clusters.

In some techniques, setting the number of clusters to 10 results in stability for all three methods. Further, these findings highlight the higher effectiveness of GMM clustering and provide valuable insights for selecting appropriate clustering techniques.

Once clusters parameters are defined, the next stage in the CCS may be clusters calibration. After conducting the calibration process as described above, measured ground plots may be assigned for each category and a distinct separation in tCO2e levels may be identified.

FIG. 14 depicts a Scatter plot of tCO2e distributions for measured ground plots from two rounds, round one (cross marks) and round two (triangles), assigned into different categories based on their calculated features, including the optimal Cluster Calibration Values (CCV) (circles) as described above. The plot enables the identification of specific tCO2e ranges associated with each category.

FIG. 14 illustrates that the register covers a diverse range of carbon sequestrations. Furthermore, some categories of carbon values co-exist in a same range with other categories. This is because several forest types exhibit the same carbon sequestration value, e.g. high trees with low coverage area might have the same carbon sequestration as low trees and high coverage. The data shown in this figure represent the ability of the proposed method to separate such forest types.

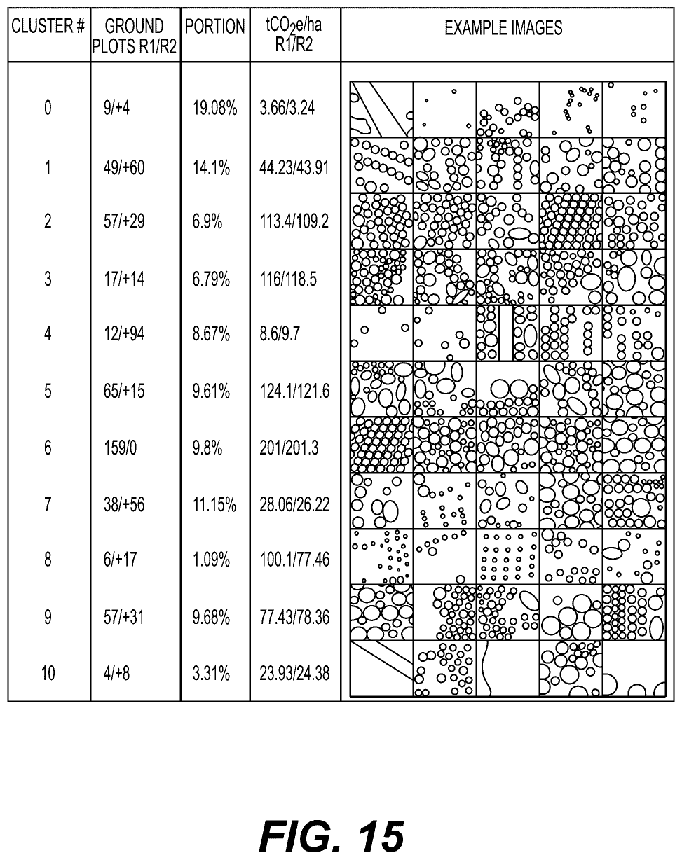

FIG. 15 illustrates a more in-depth view of the categories found by the CCS. Two categories (numbered 8 and 10) may be assigned with a low number of ground plots, and thus are defined as unstable categories. It is also shown that their proportion within the study area is very low. Apart from those, all other categories exhibit similar proportions, thus each may be interpreted as a distinct forest type. This also can be seen through the example patch images presented, where in addition to carbon-wise similarity, those forest areas exhibit similar visual properties. This is also expected as these clusters display low HV values, thus having a small in-cluster feature-wise variance. As described elsewhere with regard to in the calibration process each category is assigned with mean category error err_{c}, calculated on the test group.

The information in FIG. 15 is illustrative of different clusters and their respective ground plots quantities, including predicted CO2 value for R1 and R2, portion across the study area, and examples in each category of images for reference. It should be noted that remote data collection sourced for row 10 of in FIG. 15 may have extended beyond bounds for these techniques, e.g., portions of the images that depict tilled fields. In some embodiments, the training area may be clipped or pruned to only include data from the desired bounds, which may result in such data not being included.

Using the set of ground plots collected during the second round, as described above, it is possible to address the calibration needs of unstable categories that initially had fewer than, for example, 10 available ground plots. By adding representative ground plots from this round, the error for category #10 may be reduced and the confidence level for both categories #8 and #10 increased. Furthermore, this iterative process of multiple rounds enables refinement of the remaining categories, as depicted in FIG. 15.

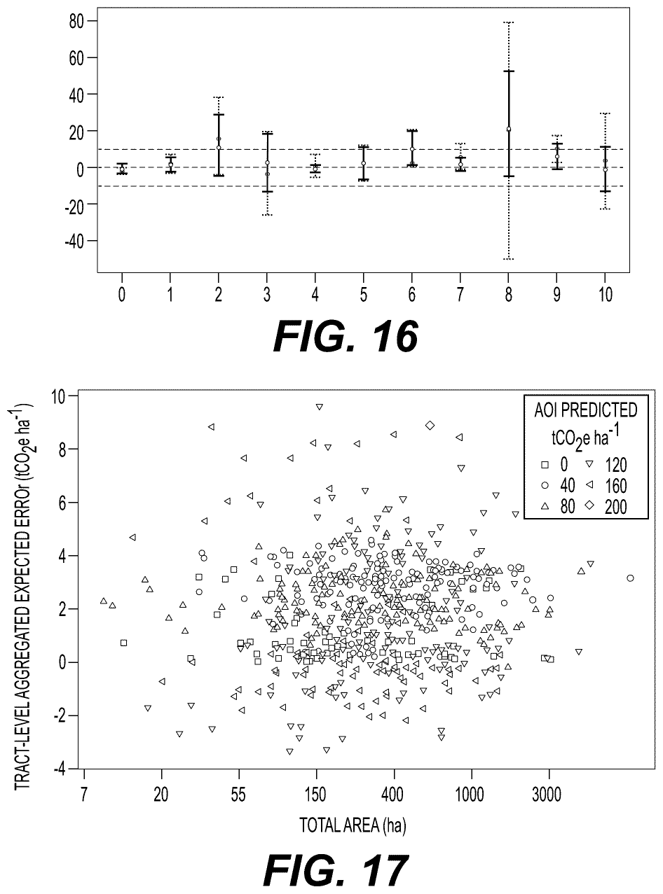

FIG. 16 depicts the tCO2e/ha errors (vertical axis) by category (horizontal axis), per Sampling Iterations (first round in solid lines, second round in dashed lines), with respective 95% Confidence Intervals. Notably, previously unstable categories #8 and #10 present lower error intervals after receiving additional plots due to the iterations, while the other categories demonstrate lower errors and error intervals, indicating improved accuracy.

In various embodiments, approaches to carbon sequestration estimation may employ calibrated categories that are tailored for small areas, and may incorporate hierarchical concepts such as patches, private lands, and projects. This may enable estimation of carbon sequestrations at different scales and assessment of estimation accuracy across various geographical and business components.

For each of the 657 tracts within the study area, the total aggregated carbon sequestration and the aggregated expected error were calculated. The analysis reveals a mean expected error rate of 1.9 tCO2e/ha with a maximum of 9.6 tCO2e/ha within a tract area, which ranges from 9.57 to 8648 hectares.

FIG. 17 depicts a scatter-plot of expected error per area of interest (AOI) in tCO2e/ha (vertical axis), lands area in hectares (horizontal axis), with the legend 1602 illustrating predicted tCO2e as a function of tract area.

To examine the spatial distribution of categories, an analysis of the distribution of patches within a representative tract may be performed. By utilizing aerial imagery, the tract may be divided into distinct continuous forest areas, each corresponding to its specific carbon sequestration. The presence of these distinct yet continuous areas is a logical characteristic of contiguous entities like forests, where scattered forest types are less prevalent.

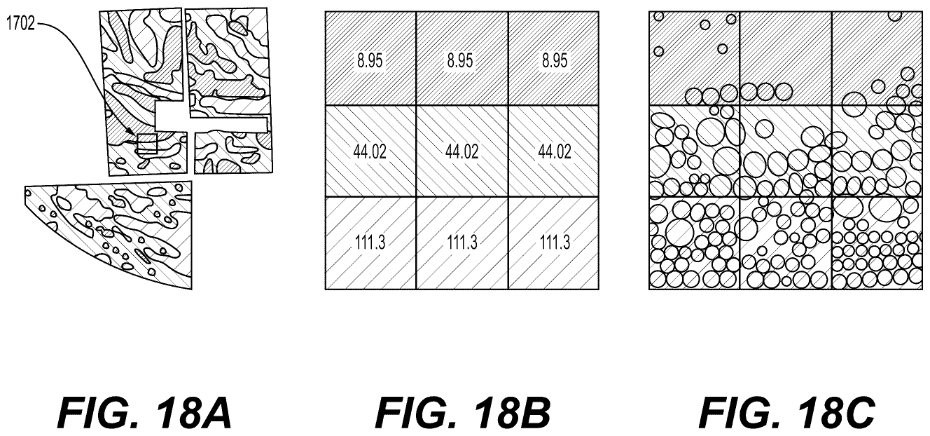

FIGS. 18A-C depict a Case study review. FIG. 18A illustrates the application of categorization to a specific area, dividing it into distinct groups (categories). The region 1802 indicates a mixed sub-area for detailed examination. FIG. 18B presents a detailed view of the region 1802, and includes assigned tCO2e/ha for each sub-area. FIG. 18C illustrates the areal images corresponding to the RGB images of the region 1802.

In an exemplary embodiment, an additional output that may be produced via one or more of the techniques disclosed herein accounts for a range of uncertainty of the model, and addresses problematic AOIs. For GMM clustering, a probability density function may be selected as an uncertainty measure for assigning a new sample to a category. Patches with a predetermined category-assignment probability of, for example, 0.5 or less for the dominant category may be considered uncertain. By analyzing the entire collection of patches in the study area, it may be found that uncertain patches constitute 1% of the total data. Such patches can include flooded areas, roads, forest gaps, and undeveloped forest areas.

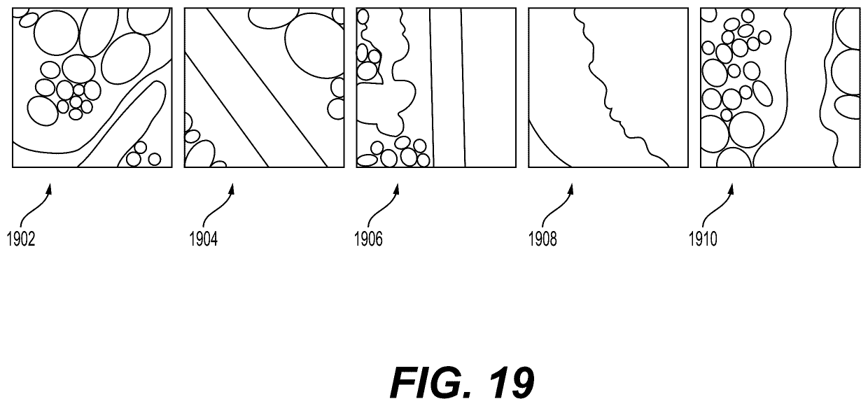

FIG. 19 depicts examples of the 1% uncertain patches extracted from the data using the category method. The picture represents (from left to right) a forest block edge with a water puddle 1902, an asphalt road 1904, dead trees 1906, a river bank 1908, and a flooded forest area 1910. All of the foregoing are generally different from the training data, and thus received a low probability of assignment to any cluster. In some embodiments, such as embodiments in which data collection is limited to the bounds for the study, such data might not be collected. In some embodiments, training data may be collected that includes non-forested area, e.g., roads, fields, etc., such that the training accounts for that type of terrain.

In embodiments herein, the capabilities and potential of the CCS technique are demonstrated, illustrating that such techniques may improve the accuracy and efficiency of forest AGB estimations worldwide. The rapid rise in carbon credit projects worldwide may lead to inaccurate accounting for AGB change, mainly stemming from the limitations of the existing methods to capture AGB changes in large, diverse, and/or complex forest ecosystems. The results of these techniques, verified through the large-scale, comprehensive ground and airborne surveys of a complex forest, emphasize the importance of using the CCS approach as a successful tool for estimating AGB.

The features presented in Table 1 and FIG. 16 are a good representation of AGB-related information which might only be obtained only from high granularity, multispectral and LiDAR measurements. Having such a list of features allows refinement and optimization of the most suitable collection of attributes tailored to the distinct traits and needs of diverse ecosystems. The feature selection process, using both RF and PCA approaches improved the accuracy of the analysis and enabled identification of the most relevant features for each cluster that should be included in the register (e.g., with reference to FIGS. 13 and 14). The CCS's ability to segment the entire area of a mixed heterogeneous forest (Table 1) and homogenize it has resulted in high-resolution CO2e prediction by category (FIG. 16), can help fine-tune carbon estimation at different AOI scales. In some embodiments, this iterative analysis may result in different features being identified as corresponding to aboveground carbon sequestration in different forests.

The features may be classified into four categories; Height, Texture, Spectral, and Intensity. As illustrated in some embodiments above, tree height may be a good indicator in allometric relations to DBH and biomass, together with crown diameter and canopy dimensions and may thus represent a straightforward value that can be obtained from LiDAR measurements.

Texture, while more vaguely defined, may be usable to identify the homogeneity of a patch, expressing the patch's internal variability, e.g., trees vs shrubs or small and big trees. Spectral features highlight a utility of plant vividness and activity as a proxy of biomass, especially biomass dynamic and tree health. By incorporating the spectral features, the accuracy of AGB estimation may be improved by considering the greenness and chlorophyll content of the vegetation in addition to the structural information provided by LiDAR and multispectral imagery. Further, the intensity, representing the points cloud are indicative of intensity (which may be usable as a proxy of canopy cover), and biomass stored in the leaves (which is supported by the stem). In the embodiment illustrated above, all of the features in Table 3 are a result of an unsupervised clustering (beside the ‘sum-average’), that emerged as a repetitive pattern from the forest characteristics. Hence, each category, in embodiments, may be based on a combination of dominant forest sub-types when using CCS. This will allow better estimation of AGB in different types of forests and vegetation, and may account for the variations in AGB due to changes in vegetation health, productivity, and species composition.

One advantage of using remote sensing data for AGB estimations of large areas is the ability to capture and identify areas that are uncharacteristic of the general AOIs. The identification of uncertain areas (FIG. 19), may enable a higher confidence in the AGB prediction and may reduce a risk of carbon overestimation and improve accuracy of carbon crediting. Ground surveys alone may overlook such areas and overestimate their AGB contribution. Similarly, using simple remote sensing methods without clustering these areas into low or uncertain carbon categories can lead to large inaccuracies, especially when sampling large forested areas via airborne methods or satellites. Therefore, a strict approach may be used, in which these areas may be used to segment new categories if there are sufficient areas (>1%) that can be aggregated into a separate new category. If there are insufficient areas (<1%), entire uncertain patches may be omitted from the total calculation, reducing a risk of overestimation of carbon in a given AOI. The CCS's capacity to optimize features' weights, self-calibrate, and create new categories may improve the accuracy of AGB estimations in uncertain areas and reduces errors in repeated measurements at the same or similar AOI category. If additional validation is required, the ground truth team can measure those specific uncertain areas that were identified by the CCS and measure their AGB.

The calibration process for each category yields a remarkably low error rate within the register, with the example techniques above exhibiting a mean and standard deviation of 2.66 and 24.4 tCO2e/ha, respectively. In comparison to other published methods that utilize satellite data or a combination of satellite and aerial data, the CCS technique demonstrates a higher level of agreement with ground survey data collected from mixed forests and exhibits lower root mean square error (RMSE) values. In one embodiment, the combination of large sample size and low RMSE suggests that the CCS technique may provide improved accuracy and utility. The aggregation analysis depicted in FIG. 17 examines the anticipated sequestration error, measured in tCO2e/ha, across various area of interest (AOI) sizes. The analysis reveals that low-carbon tracts tend to have lower errors compared to higher-carbon tracts. Furthermore, the technique demonstrates relatively consistent accuracy across a range of aggregation areas, providing high accuracy for both small-sized and large-sized AOIs.

By applying this approach at the level of private land, a method according to one or more aspects of this disclosure offers unique insights that can provide value to landowners and management entities. Such techniques may provide valuable tools to quantify and monetize their contributions to carbon sequestration, promoting sustainable land management practices on a smaller scale that was not previously feasible.

While the Register-based Carbon Sequestration (CCS) scheme offers numerous benefits, it is important to understand the necessary criteria and potential limitations associated with its implementation. For example, the establishment of stable categories and a register may involve the collection of extensive aerial data, including LIDAR and multispectral imagery, as well as ground-based measurements of tree dimensions.

Furthermore, since the clusters may be created using unsupervised learning, there may be instances where certain clusters are not well-defined or easily explainable. In some embodiments, for example if explainability is a priority, an additional stage of supervised learning may be incorporated to retain only logical clusters while filtering out others. Additionally, as described above, to maintain accurate carbon sequestration, it is may be beneficial to have more than 10, or more than 30, ground plots for each category (predetermined values may vary). This tradeoff between the number of categories and the accuracy of each category may be considered during the implementation of the method. Moreover, in order to estimate carbon sequestration additionality, a considerable number of categories may be needed, e.g., in the order of several tens. This may reduce the data efficiency of the proposed method, as a larger number of categories can lead to increased computational and data processing requirements.

The ability to link remote-sensing data inputs and allometric relationships in various ecosystems and forest types is a major improvement in estimating the AGB of large and complex areas. Satellite data, with its availability and low cost, is valuable for global forests' biomass estimation, but generally has a low resolution (10 to 30 m) which may hinder accuracy and granularity. To utilize the demonstrated capabilities of the CCS, high quality LiDAR or RGB data and ground-truthing measurements may be used. By combining CCS with satellite and airborne data, the AOI can be filtered and categorized, with airborne campaigns focused on areas that require higher resolution measurements, identified by the CCS. This combined dataset can then be validated on the ground to represent the relevant AOIs. One advantage of the CCS technique is the reduced reliance on airborne and ground truth samples, improved efficiency, and reduced costs. Identifying relevant areas suitable for airborne measurement through satellite data categorization results in cost, effort, and more efficient quantification of aboveground biomass carbon sequestration.

The majority of studies aiming to improve AGB estimation by RS and ML produce a general regression between the predicted and measured AGB for the entire study area, resulting in low to moderate coefficients. This may be due to the fact that the correlation may be done on the entire dataset without consideration of the forest complexity, heterogeneity, and uncertainties. By adding the CCS approach to these datasets, segmented regressions can be generated, tailored for each category, leading to higher coefficients and improved predictions and increasing the accuracy of prediction, and improving the upscaling potential of the RS measurement of complex ecosystems.

In some embodiments, one or more aspects of DL may be combined with one or more aspects of the feature extraction/selection protocol discussed above. Conventionally, the feature extraction is done by “feature engineering”. In an embodiment, DL may be utilized to discover patterns that may go unnoticed through traditional feature engineering methods. These patterns can be derived from both supervised and unsupervised DL approaches. By incorporating DL, it is possible to leverage a broader range of features, including more robust and stable ones. However, it is important to note that explaining the rationale behind DL-identified features may be challenging compared to engineered features. Therefore, a combination of DL and feature engineering approaches can be employed to strike a balance between capturing complex patterns and ensuring interpretability.

The CCS mechanism described in these techniques may be configured to automatically detect unrecognized forest types, assign ground survey tasks, and use the surveyed data to recalibrate itself by finding the best features to use along with optimal carbon sequestration. The CCS technique may disrupt the current approach to forest carbon sequestration by dividing the forest area into smaller clusters representing homogeneous forest types, thereby reducing the error that arises from the natural heterogeneity and simplifying the AGB estimation process. Each cluster is calibrated using a fixed value of carbon sequestration, which also reduces the need for large, intensive, and costly ground surveys, while still achieving high accuracy.

As discussed above, such an approach has been shown to achieve high accuracy on several aggregation levels, unlocking new opportunities for the carbon sequestration market for small-sized lands.

While several of the examples above involve processing electronic images to estimate carbon sequestration, it should be understood that techniques according to this disclosure may be adapted to any suitable type of image processing. It should also be understood that the examples above are illustrative only. The techniques and technologies of this disclosure may be adapted to any suitable activity.

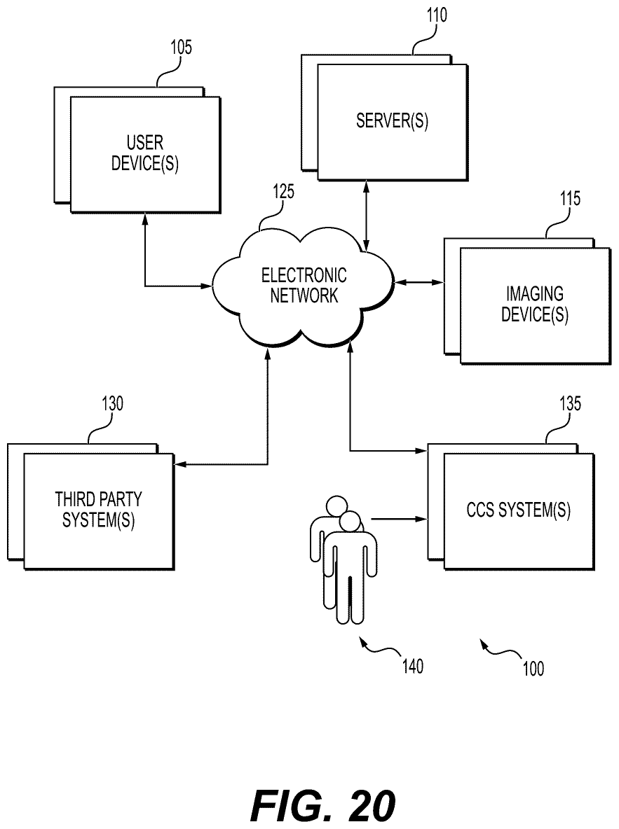

FIG. 20 illustrates an exemplary environment 100 that may be utilized with techniques presented herein. The environment 100 may include one or more user device(s) 105, one or more server(s) 110, or one or more sensor or imaging device(s) 115, which may communicate, e.g., via any suitable means such as via an electronic network 125, with each other or with any suitable third party system 130. As discussed in further detail below, the environment 100 may further include one or more CCS system(s) 135 that may be configured to execute one or more of the techniques discussed above. In some embodiments, a ground team 140 may be associated with the CCS system 135.

In some embodiments, the components of the environment are associated with a common entity, e.g., a carbon credit tracking entity, a land surveyor, or the like. In some embodiments, one or more of the components of the environment 100 is associated with a different entity than another. The systems and devices of the environment 100 may communicate in any arrangement. Further, various systems and/or devices of the environment 100 may communicate in order to one or more of generate, train, or use a machine-learning model as discussed above.

The user device 105 may be configured to enable a user to access and/or interact with other systems in the environment 100. For example, the user device 105 may be a computer system such as, for example, a desktop computer, a mobile device, a tablet, etc. In some embodiments, the user device 105 may include one or more electronic application(s), e.g., a program, plugin, browser extension, etc., installed on a memory of the user device. In some embodiments, the electronic application(s) may be associated with one or more of the other components in the environment. For example, the electronic application(s) may include one or more of system control software, system monitoring software, software development tools, etc.

A server 110 may include, for example, an electronic data system, computer-readable memory such as a hard drive, flash drive, disk, etc. In some embodiments, the server 110 includes and/or interacts with an application programming interface for exchanging data to other systems, e.g., one or more of the other components of the environment. The server 110 may include and/or act as a repository or source for data.

An imaging device 115 may include, for example, a satellite imaging device, an aerial imaging device such as a piloted aircraft or autonomous aircraft, or the like. A third party system 130, as used to herein, generally encompasses any system that a component in the environment 100 may communicate with in the course of operation. In an example, a third party system may include a system associated with an entity or data store, or the like that utilizes an imaging device 115 to capture images. Another example of a third party system may include a data store that includes data regarding a region, e.g., environmental data, tree data, etc.

In various embodiments, the electronic network 125 may be a wide area network (“WAN”), a local area network (“LAN”), personal area network (“PAN”), or the like. In some embodiments, electronic network 125 includes the Internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks—a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

The CCS system 135 may include the hardware and software usable to execute one or more of the techniques disclosed above. In an example, the CCS system 135 may include a computer system configured to communicate with other components of the environment 100, the computer system having one or more model for implementing a CCS scheme, data usable by the CCS system 135 to Although certain examples of machine learning models were discussed in examples above, it should be understood that any suitable type of machine learning model or combination of models or techniques may be employed.

It should be understood that a component or portion of a component in the environment 10 may, in some embodiments, be integrated with or incorporated into one or more other components. In some embodiments, operations or aspects of one or more of the components discussed above may be distributed amongst one or more other components. Any suitable arrangement and/or integration of the various systems and devices of the environment may be used.

It should be understood that embodiments in this disclosure are exemplary only, and that other embodiments may include various combinations of features from other embodiments, as well as additional or fewer features.

In general, any process or operation discussed in this disclosure that is understood to be computer-implementable may be performed by one or more processors of a computer system, such any of the systems or devices in the environment, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices, such as one or more of the systems or devices in the environment. One or more processors of a computer system may be included in a single computing device or distributed among a plurality of computing devices. A memory of the computer system may include the respective memory of each computing device of the plurality of computing devices.

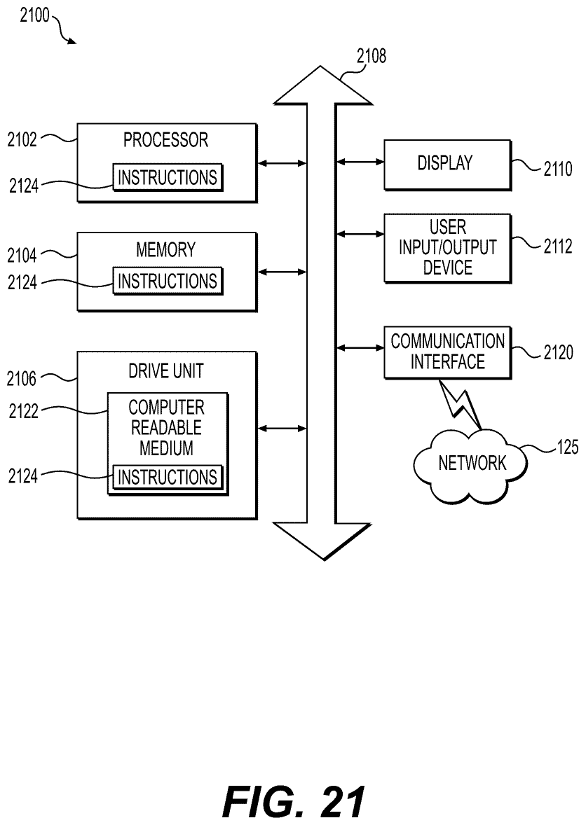

FIG. 21 is a simplified functional block diagram of a computer 2100 that may be configured as a device for executing one or more aspects of the present disclosure.

In various embodiments, any of the systems herein may be a computer 2100 including, for example, a data communication interface 2120 for packet data communication. The computer 2100 also may include a central processing unit (“CPU”) 2102, in the form of one or more processors, for executing program instructions. The computer 2100 may include an internal communication bus 2108, and a storage unit 2106 (such as ROM, HDD, SDD, etc.) that may store data on a computer readable medium 1922, although the computer 1900 may receive programming and data via network communications. The computer 2100 may also have a memory 2104 (such as RAM) storing instructions 2124 for executing techniques presented herein, although the instructions 2124 may be stored temporarily or permanently within other modules of computer 2100 (e.g., processor 2102 and/or computer readable medium 2122). The computer 2100 also may include input and output ports 2112 and/or a display 2110 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the disclosed methods, devices, and systems are described with exemplary reference to transmitting data, it should be appreciated that the disclosed embodiments may be applicable to any environment, such as a desktop or laptop computer, etc. Also, the disclosed embodiments may be applicable to any type of Internet protocol.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the invention requires more features than are expressly recited in an individual embodiment. Rather, inventive aspects lie in less than all features of a single foregoing disclosed embodiment.

Furthermore, while some embodiments described herein include some but not other features included in other embodiments, combinations of features of different embodiments are meant to be within the scope of the invention, and form different embodiments, as would be understood by those skilled in the art.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the disclosure is intended to cover all such modifications, enhancements, and other implementations, which fall within the true spirit and scope of the present disclosure. While various implementations of the disclosure have been described, it will be apparent to those of ordinary skill in the art that many more implementations are possible within the scope of the disclosure.

What is claimed is:

1. A computer-implemented method for registering carbon sequestration, comprising:
receiving one or more entries of carbon sequestration data, each entry corresponding to a respective portion of a geographic region;
comparing each entry to a feature space in a register of entries for the geographic region;
based on the comparing, assigning each entry to a respective cluster amongst a plurality of clusters of entries in the register, each cluster of the plurality of clusters being associated with a respective carbon sequestration value; and
determining a total carbon sequestration for the geographic region based on an aggregating of each carbon sequestration value of each entry in the geographic region.

2. The computer-implemented method of claim 1, wherein the aggregating comprises:
aggregating each entry corresponding to a respective sub-region of the geographic region;
determining a sub-region carbon sequestration value for each sub-region; and
determining the total carbon sequestration for the geographic region based on an aggregation of the sub-region carbon sequestration value for each sub-region.

3. The computer-implemented method of claim 1, further comprising:
based on the comparing, determining a respective confidence for the assigning.

4. The computer-implemented method of claim 3, further comprising:
aggregating each entry corresponding to a respective sub-region of the geographic region; and
determining a proportion of entries in each sub-region having at least a threshold confidence.

5. The computer-implemented method of claim 4, further comprising:
in response to determining that the proportion of entries in a sub-region is below a further threshold, causing at least one imaging device to capture further new entries of one or more portion of the sub-region;
updating the register using the further new entries; and
iterating the method using the updated register.

6. The computer-implemented method of claim 5, wherein:
the at least one imaging device includes an autonomous aerial vehicle; and
the causing of the at least one imaging device to capture the further new entries includes transmitting an instruction to the autonomous aerial vehicle configured to cause the autonomous aerial vehicle to travel to the one or more portion of the sub-region and capture the further new entries.

7. The computer-implemented method of claim 1, wherein the plurality of clusters and the respective carbon sequestration values have been determined via a trained machine-learning model that has been trained based on carbon sequestration data from entries form the register.

8. The computer-implemented method of claim 1, wherein each entry includes, for the corresponding portion of the geographic region, one or more of multispectral data, LIDAR point cloud data, canopy height model data, or features determined therefrom.

9. The computer-implemented method of claim 8, further comprising:
preprocessing the LiDAR point cloud data by:
segmenting the LiDAR point cloud data into voxels, each voxel represented by a value corresponding to a number of pixels associated with each voxel;
determining one or more connected component of voxels;
determining angles between a center of mass of each connected component and a largest connected component;
pruning points in the LiDAR point cloud data corresponding to connected components having an angle above a predetermined threshold;
performing a height normalization of remaining points in the LiDAR point cloud data.

10. The computer-implemented method of claim 8, further comprising:
generating canopy height model data based on the LiDAR point cloud data.

11. A computer-implemented method for registering carbon sequestration, comprising:
receiving one or more entries of carbon sequestration data, each entry corresponding to a respective portion of a geographic region, the geographic region including at least one sub-region, and each sub-region including at least one portion;
comparing each entry to a feature space in a register of entries for the geographic region;
based on the comparing, assigning each entry to a respective cluster amongst a plurality of clusters of entries in the register, each cluster of the plurality of clusters being associated with a respective carbon sequestration value;
for each sub-region in the geographic region:
aggregating all entries for each portion included in the sub-region; and
determining a respective carbon sequestration value for the sub-region based on the respective carbon sequestration values assigned to each entry aggregated for the sub-region; and
determining a total carbon sequestration for the geographic region based on the respective carbon sequestration value of each sub-region in the geographic region.

12. The computer-implemented method of claim 11, further comprising:
based on the comparing, determining a respective confidence for the assigning.

13. The computer-implemented method of claim 12, further comprising:
for each sub-region in the geographic region, determining a proportion of entries in each sub-region having at least a threshold confidence.

14. The computer-implemented method of claim 13, further comprising:
in response to determining that the proportion of entries in at least one sub-region is below a further threshold, causing at least one imaging device to capture further new entries of one or more portion of the at least one sub-region;
updating the register using the further new entries; and
iterating the method using the updated register.

15. The computer-implemented method of claim 14, wherein:
the at least one imaging device includes an autonomous aerial vehicle; and
the causing of the at least one imaging device to capture the further new entries includes transmitting an instruction to the autonomous aerial vehicle configured to cause the autonomous aerial vehicle to travel to the one or more portion of the sub-region and capture the further new entries.

16. The computer-implemented method of claim 11, wherein the plurality of clusters and the respective carbon sequestration values have been determined via a trained machine-learning model that has been trained based on carbon sequestration data from entries form the register.

17. The computer-implemented method of claim 11, wherein each entry includes, for the corresponding portion of the geographic region, one or more of multispectral data, LIDAR point cloud data, canopy height model data, or features determined therefrom.

18. The computer-implemented method of claim 17, further comprising:
preprocessing the LiDAR point cloud data by:
segmenting the LiDAR point cloud data into voxels, each voxel represented by a value corresponding to a number of pixels associated with each voxel;
determining one or more connected component of voxels;
determining angles between a center of mass of each connected component and a largest connected component;
pruning points in the LiDAR point cloud data corresponding to connected components having an angle above a predetermined threshold;
performing a height normalization of remaining points in the LiDAR point cloud data.

19. The computer-implemented method of claim 17, further comprising:
generating canopy height model data based on the LiDAR point cloud data.

20. A system for registering carbon sequestration, comprising:
at least one memory storing instructions and a register of entries of carbon sequestration data for a geographical region, the geographic region including at least one sub-region, and each sub-region including at least one portion, each entry corresponding to a respective portion of the geographic region;
at least one processor operatively connected to the at least one memory, and configured to execute the instructions to perform operations, including:
assigning each entry to a respective cluster amongst a plurality of clusters of entries in the register, each cluster of the plurality of clusters being associated with a respective carbon sequestration value;
for each sub-region in the geographic region:
aggregating all entries for each portion included in the sub-region; and
determining a respective carbon sequestration value for the sub-region based on the respective carbon sequestration values assigned to each entry aggregated for the sub-region; and
determining a total carbon sequestration for the geographic region based on the respective carbon sequestration value of each sub-region in the geographic region.
